(19) United States
(12) Patent Application Publication
(10) Pub. No.: US 2022/0347313 A1
(43) Pub. Date: Nov. 3, 2022

(54) Combination Anti-CD30 ADC, Anti-PD-1 and Chemotherapeutic for Treatment of Hematopoietic Cancers

(71) Applicant: SEAGEN, INC., Bothell, WA (US)

(72) Inventor: Thomas Manley, Bothell, WA (US)

(21) Appl. No.: 17/762,867

(22) PCT Filed: Sep. 25, 2020
(86) PCT No.: PCT/US20/52674

§ 371 Date: Mar. 23, 2022

Related U.S. Application Data
(60) Provisional application No. 62/905,701, filed on Sep. 25, 2019.

(51) Int. Cl. A61K 47/68; A61P 35/00; A61K 39/395; A61K 31/704; A61K 31/655; A61K 38/19

(52) U.S. Cl. CPC A61K 47/6867; A61P 35/00; A61K 47/6849; A61K 39/39558; A61K 31/704; A61K 31/655; A61K 38/193; A61K 2039/54

(57) ABSTRACT

The present disclosure relates, in general to methods for treating hematologic cancers comprising administering an anti-CD30 antibody drug conjugate in combination with additional cancer therapeutics such as a checkpoint inhibitor, and a chemotherapeutic regimen.

### CROSS-REFERENCE TO RELATED APPLICATIONS

The present application is a national stage of PCT/US2020/52674, filed Sep. 25, 2020 which claims the priority benefit of U.S. Provisional Patent Application No. 62/905,701, filed Sep. 25, 2019, the disclosures of which are hereby incorporated by reference in their entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “54784_Seqlisting.txt”, which was created on Sep. 24, 2020 and is 7,899 bytes in size. The subject matter of the Sequence Listing is incorporated herein in its entirety by reference.

### FIELD OF THE DISCLOSURE

The present disclosure relates, in general, to methods of treating Hodgkin's lymphoma and other hematopoietic cancer with anti-CD30 antibody drug conjugate therapy in combination with anti-PD-1 antibody, and optionally in combination with a chemotherapeutic regimen comprising doxorubicin and dacarbazine.

### BACKGROUND

Outcomes for patients with advanced-stage Hodgkin lymphoma have improved dramatically over the past half century.¹ Although regional differences exist, the most commonly used frontline regimen, ABVD (doxorubicin, bleomycin, vinblastine, and dacarbazine), has not been modified since its original description in 1975.

Up to 30% of patients with stage III/IV Hodgkin lymphoma harbor refractory disease or relapse following frontline ABVD (Canellos et al., N Engl J Med 1992; 327:1478-84; Carde et al., J Clin Oncol 2016; 34:2028-36; Gordon et al., J Clin Oncol 2013; 31:684-91). Bleomycin, considered to have the least activity of the four components of ABVD, is associated with unpredictable and sometimes fatal pulmonary toxicity, and is often dropped from later cycles of chemotherapy due to pulmonary symptoms (Canellos et al., J Clin Oncol 2004; 22:1532-3; Martin et al., J Clin Oncol 2005; 23:7614-20). Recent studies suggest that response-adapted therapy guided by interim positron-emission tomography (PET) with 18F-fluorodeoxyglucose can provide a more individualized treatment approach in which treatment intensity is de-escalated/intensified depending on the early response to treatment (Borchmann et al., Haematologica 2017; 102:Abstract S150; Johnson et al., N Engl J Med 2016; 374:2419-29). Efforts are also being made to incorporate new drugs into established backbones to improve efficacy and reduce toxicity (Borchmann et al., Blood 2015; 126).

CD30 is a characteristic surface antigen expressed on the Reed-Sternberg cells of classical Hodgkin lymphoma (Schwab et al., Nature 1982; 299:65-7). Brentuximab vedotin is an antibody-drug conjugate composed of an anti-CD30 monoclonal antibody conjugated by a protease-cleavable linker to the microtubule disrupting agent, monomethyl auristatin E. Brentuximab vedotin has been approved for the treatment of classical Hodgkin lymphoma patients after failure of autologous stem cell transplant (ASCT) or after failure of at least 2 prior multi-agent chemotherapy regimens in patients who are not ASCT candidates, and as consolidation post-ASCT for Hodgkin lymphoma patients at increased risk of relapse/progression (ADCETRIS® (brentuximab vedotin) US Prescribing Information). It has also been approved for systemic anaplastic large cell lymphoma after failure of at least one prior multi-agent chemotherapy regimen. A previous phase 1, dose-escalation study in advanced Hodgkin lymphoma evaluated frontline brentuximab vedotin combined with either ABVD or AVD (doxorubicin, vinblastine, dacarbazine) (Younes et al., Lancet Oncol 2013; 14:1348-56).

Nivolumab is a fully-humanized monoclonal antibody (HuMAb; immunoglobulin G4 [IgG4]) that targets programmed cell death protein 1 (PD-1). In vitro, nivolumab binds to PD-1 with high affinity and inhibits the binding of PD-1 to its ligands PD-L1 and PD-L2 (IC50±1 nM). Nivolumab binds specifically to PD-1 and not to related members of the CD28 family such as CD28, ICOS, CTLA-4 and BTLA. Nivolumab blocks the PD-1 pathway and results in a reproducible enhancement of both proliferation and interferon-gamma (IFN-γ) release in the mixed lymphocyte reaction (MLR). Previous trials have shown that treatment of patients with refractory Hodgkin Lymphoma with a combination of brentuximab vedotin and nivolumab is safe and patients were able to undergo subsequent stem cell transplant (Herrera et al., Blood 131(11): 1183-94.) The combination of nivolumab has also been shown to be well tolerated when combined with doxorubicin, vinblastine, and dacarbazine (N+AVD) (Ramchandren et al., J Clin Oncol 37(23): 1997-2007).

### SUMMARY OF THE DISCLOSURE

The present disclosure relates to methods of treating a hematologic cancer comprising administering an anti-CD30 antibody drug conjugate (ADC) in combination with cancer therapeutics as a front line treatment for previously undiagnosed cancers, or as a treatment for relapsed or refractory disease. Additional cancer therapeutics used in combination with the anti-CD30 ADC include anti-PD-1 antibodies and a chemotherapeutic regimen comprising doxorubicin and dacarbazine.

Provided herein is a method for treating a hematologic cancer in a subject comprising administering a therapy comprising an anti-CD30 antibody drug conjugate and an anti-PD-1 antibody, doxorubicin and dacarbazine.

In various embodiments, the anti-PD-1 antibody is administered at least 30 minutes after each administration of anti-CD30 antibody drug conjugate. In various embodiments, the anti-CD30-antibody drug conjugate is administered by intravenous infusion over a period of about 30 minutes. In various embodiments, the anti-PD-1 antibody is administered by intravenous infusion for a duration of approximately 60 minutes.

In various embodiments, the anti-PD-1 antibody is administered to a subject that has not received anti-CD30 antibody drug conjugate therapy previously. In various embodiments, the anti-CD30 antibody drug conjugate is administered to a subject that has not received anti-CD30 antibody drug conjugate therapy previously. In various embodiments, the subject has not received checkpoint inhibitor therapy or an anti-PD-1 antibody previously.

In various embodiments, the anti-CD30 antibody drug conjugate and anti-PD-1 antibody are administered every 2 weeks. In various embodiments, the anti-PD-1 antibody is administered beginning with cycle 1 of the administration of anti-CD30 antibody drug conjugate. In various embodiments, the anti-CD30 antibody drug conjugate and anti-PD-1 antibody are administered on day 1 and day 15 of a 28-day cycle. In various embodiments, the anti-CD30 antibody drug conjugate and anti-PD-1 antibody are administered for no more than six cycles. In various embodiments, the anti-CD30 antibody drug conjugate and anti-PD-1 antibody are administered for four to six cycles.

In various embodiments, the method further comprises administering a chemotherapy consisting essentially of doxorubicin and dacarbazine (AD) as a combination therapy.

In various embodiments, the anti-CD30 antibody of the anti-CD30 antibody drug conjugate comprises i) a heavy chain CDR1 set out in SEQ ID NO: 4, a heavy chain CDR2 set out in SEQ ID NO: 6, a heavy chain CDR3 set out in SEQ ID NO: 8; and ii) a light chain CDR1 set out in SEQ ID NO: 12, a light chain CDR2 set out in SEQ ID NO: 14, and a light chain CDR13 set out in SEQ ID NO: 16.

In various embodiments, the anti-CD30 antibody of the anti-CD30 antibody drug conjugate comprises i) an amino acid sequence at least 85% identical to a heavy chain variable region set out in SEQ ID NO: 2 and ii) an amino acid sequence at least 85% identical to a light chain variable region set out in SEQ ID NO: 10.

In various embodiments, the anti-CD30 antibody of the anti-CD30 antibody drug conjugate is a monoclonal anti-CD30 antibody. In various embodiments, the anti-CD30 antibody of the anti-CD30 antibody drug conjugate is a chimeric AC10 antibody. In various embodiments, the antibody drug conjugate comprises monomethyl auristatin E and a protease-cleavable linker. In various embodiments, the protease cleavable linker is comprises a thiolreactive spacer and a dipeptide. In various embodiments, the protease cleavable linker consists of a thiolreactive maleimidocaproyl spacer, a valine—citrulline dipeptide, and a p-amino-benzyloxycarbonyl spacer. In various embodiments, the anti-CD30 antibody drug conjugate is brentuximab vedotin.

In various embodiments, the anti-PD-1 antibody is a monoclonal antibody. In various embodiments, (i) the anti-PD-1 antibody cross-competes with nivolumab or pembrolizumab for binding to human PD-1; (ii) the anti-PD-1 antibody binds to the same epitope as nivolumab or pembrolizumab; (iii) the anti-PD-1 antibody is nivolumab; or (iv) the anti-PD-1 antibody is pembrolizumab. In various embodiments, the anti-PD-1 antibody is nivolumab or pembrolizumab. In various embodiments, the anti-PD-1 antibody is nivolumab.

In various embodiments, the hematologic cancer comprises one or more cells that express PD-L1, PD-L2, or both PD-L1 and PD-L2.

In various embodiments, the hematologic cancer is a CD30-expressing cancer and the CD30 expression is ≥10. In various embodiments, the CD30 expression is measured by a FDA approved test.

In various embodiments, the anti-CD30 antibody drug conjugate is brentuximab vedotin and is administered at 1.2 mg/kg, and the anti-PD-1 antibody is nivolumab and is administered at 240 mg/dose.

In various embodiments, the anti-CD30 antibody drug conjugate is brentuximab vedotin and is administered at 1.2 mg/kg, and the anti-PD-1 antibody is pembrolizumab and is administered at a dose of 1-2 mg/kg, or 100-300 mg.

In various embodiments, doxorubicin is administered at dose of 25 mg/m², and dacarbazine is administered at a dose of 375 mg/m².

In various embodiments, the method further comprises administering a granulopoiesis stimulating factor. In various embodiments, the granulopoiesis stimulating factor is administered from 1 day to 7 days after administration of anti-CD30 antibody drug conjugate. In various embodiments, the granulopoiesis stimulating factor is administered from 2 days to 5 days after the administration of anti-CD30 antibody drug conjugate. In various embodiments, the granulopoiesis stimulating factor is administered about 24 hours to about 36 hours after administration of anti-CD30 antibody drug conjugate.

In various embodiments, the granulopoiesis stimulating factor is a granulocyte-colony stimulating factor (GCSF). In various embodiments, the GCSF is a long-acting GCSF or a non long-acting GCSF. In various embodiments, the GCSF is long-acting GCSF, and is administered 1 day or 2 days after the administration of anti-CD30 antibody drug conjugate. In various embodiments, the G-CSF is administered about 24 hours to about 36 hours after administration of anti-CD30 antibody drug conjugate. In various embodiments, the GCSF is not long acting, and is administered 1, 2, 3, 4, 5, 6 or 7 days after the administration of anti-CD30 antibody drug conjugate.

In various embodiments, the granulopoiesis stimulating factor is administered in a dose range from 5 to 10 mcg/kg/day, or 300 to 600 mcg/day, or 6 mg/dose.

In various embodiments, the granulopoiesis stimulating factor is administered to a subject that has not received anti-CD30 antibody drug conjugate therapy previously.

In various embodiments, the subject has not experienced treatment-emergent grade 3-4 neutropenia after anti-CD30 antibody drug conjugate administration. In various embodiments, the granulopoiesis stimulating factor is given intravenously or subcutaneously. In various embodiments, the granulopoiesis stimulating factor is given in a single dose or multiple doses.

In various embodiments, if the subject exhibits Grade 3 or Grade 4 neuropathy, the administration of anti-CD30 antibody drug conjugate therapy is withheld until peripheral neuropathy decreases to Grade 2 or less and then 0.9 mg/kg anti-CD30 antibody drug conjugate therapy is administered. In various embodiments, the neuropathy is motor neuropathy or sensory neuropathy. In various embodiments, the dose of anti-CD30 antibody drug conjugate is delayed by one week if peripheral neuropathy appears, and therapy is continued when the neuropathy is resolved or determined to be Grade 1 or less.

In various embodiments, the hematologic cancer is a CD30-expressing hematologic cancer. In various embodiments, the hematologic cancer is selected from the group consisting of classical Hodgkin Lymphoma, non-Hodgkin Lymphoma, cutaneous T-cell lymphoma (CTCL), and anaplastic large cell lymphoma (ALCL).

In various embodiments, the hematologic cancer is classical Hodgkin Lymphoma. In various embodiments, the hematologic cancer is a stage IIA with bulky disease, stage IIB, stage III or stage IV classical Hodgkin Lymphoma.

In various embodiments, the anaplastic large cell lymphoma (ALCL) is a systemic anaplastic large cell lymphoma (sALCL).

In various embodiments, the cutaneous T-cell lymphoma (CTCL) is a mycosis fungoides (MF). In various embodiments, the mycosis fungoides (MF) is a CD30-positive mycosis fungoides (MF). In various embodiments, the cutaneous T-cell lymphoma (CTCL) is a primary cutaneous anaplastic large cell lymphoma (pcALCL).

In various embodiments, the hematologic cancer of the subject has not been treated with a checkpoint inhibitor. In various embodiments, the subject has received prior systemic therapy.

In various embodiments, the subject is an adult patient.

It is specifically provided herein that all aspects of the disclosure described above with the methods of treatment are applicable to the anti-CD30 antibody drug conjugate combination therapy for use in any of the indications described above.

It is understood that each feature or embodiment, or combination, described herein is a non-limiting, illustrative example of any of the aspects of the disclosure and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the disclosure. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE FIGURES

FIG. 1. Details of Brentuximab Vedotin Dose Modifications

### DETAILED DESCRIPTION

The present disclosure provides methods for treating a hematologic cancer with an anti-CD30 antibody drug conjugate in combination with an anti-PD-1 checkpoint inhibitor and a chemotherapy regimen comprising doxorubicin and dacarbazine. Not to be bound by theory, but it is hypothesized that administration of the combination therapies will result in less toxicity be removal of vincristine from typical therapeutic regimens and provide an improved outcome for patients.

### Definitions

Unless otherwise defined, all technical and scientific terms used herein have the same meaning as commonly understood by one of ordinary skill in the art to which this disclosure belongs. The following references provide one of skill with a general definition of many of the terms used in this disclosure: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991).

Each publication, patent application, patent, and other reference cited herein is incorporated by reference in its entirety to the extent that it is not inconsistent with the present disclosure.

As used herein and in the appended claims, the singular forms “a,” “and,” and “the” include plural referents unless the context clearly dictates otherwise. Thus, for example, reference to “a derivative” includes a plurality of such derivatives and reference to “a subject” includes reference to one or more subjects and so forth.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

“Treatment” refers to prophylactic treatment or therapeutic treatment or diagnostic treatment. In certain embodiments, “treatment” refers to administration of a compound or composition to a subject for therapeutic, prophylactic or diagnostic purposes.

A “prophylactic” treatment is a treatment administered to a subject who does not exhibit signs of a disease or exhibits only early signs of the disease, for the purpose of decreasing the risk of developing pathology. The compounds or compositions of the disclosure may be given as a prophylactic treatment to reduce the likelihood of developing a pathology or to minimize the severity of the pathology, if developed.

A “therapeutic” treatment is a treatment administered to a subject who exhibits signs or symptoms of pathology for the purpose of diminishing or eliminating those signs or symptoms. The signs or symptoms may be biochemical, cellular, histological, functional or physical, subjective or objective.

“Therapeutically effective amount” as used herein refers to that amount of an agent effective to produce the intended beneficial effect on health.

“AN+AD therapy” as used herein refers to treatment of a subject with an anti-CD30 antibody drug conjugate (brentuximab vedotin) as described herein in combination with an antibody to PD-1 (nivolumab), and chemotherapy consisting essentially of doxorubicin, and dacarbazine (AD therapy).

“Lymphoma” as used herein is hematological malignancy that usually develops from hyper-proliferating cells of lymphoid origin. Lymphomas are sometimes classified into two major types: Hodgkin lymphoma (HL) and non-Hodgkin lymphoma (NHL). Lymphomas may also be classified according to the normal cell type that most resemble the cancer cells in accordance with phenotypic, molecular or cytogenic markers. Lymphoma subtypes under that classification include without limitation mature B-cell neoplasms, mature T cell and natural killer (NK) cell neoplasms, Hodgkin lymphoma and immunodeficiency-associated lympho-proliferative disorders. Lymphoma subtypes include precursor T-cell lymphoblastic lymphoma (sometimes referred to as a lymphoblastic leukemia since the T-cell lymphoblasts are produced in the bone marrow), follicular lymphoma, diffuse large B cell lymphoma, mantle cell lymphoma, B-cell chronic lymphocytic lymphoma (sometimes referred to as a leukemia due to peripheral blood involvement), MALT lymphoma, Burkitt's lymphoma, mycosis fungoides and its more aggressive variant Sezary's disease, peripheral T-cell lymphomas not otherwise specified, nodular sclerosis of Hodgkin lymphoma, and mixed-cellularity subtype of Hodgkin lymphoma.

“Leukemia” as the term is used herein is a hematological malignancy that usually develops from hyper-proliferating cells of myeloid origin, and include without limitation, acute lymphoblastic leukemia (ALL), acute myelogenous leukemia (AML), chronic lymphocytic leukemia (CLL), chronic myelogenous leukemia (CML) and acute monocyctic leukemia (AMoL). Other leukemias include hairy cell leukemia (HCL), T-cell lymphatic leukemia (T-PLL), large granular lymphocytic leukemia and adult T-cell leukemia.

“Prophylactic” or “primary prophylaxis” as used herein refers to administration of an agent, such as a colony stimulating factor or granulopoiesis stimulating factor, prior to onset of neutropenia or symptoms of neutropenia in a subject. It is contemplated that prophylaxis includes administration of the granulopoeisis stimulating factor at the beginning of cycle 1 of administration of anti-CD30 conjugate therapy, or first administration of the anti-CD30-antibody drug conjugate therapy, optionally in combination with a therapy consisting essentially of anti-PD-1 antibody, doxorubicin, and/or dacarbazine (N+AD therapy). The term “beginning with cycle 1 of the administration of the anti-CD30 antibody drug conjugate” and “first administration of the anti-CD30 antibody drug conjugate” are used interchangeably herein in reference to treatment with granulopoiesis stimulating factor.

“Granulopoiesis stimulating factor” as used herein refers to an agent such as a cytokine or other growth factor that can induce production of neutrophils and other granulocytes. Exemplary granulopoiesis stimulating factors include, but are not limited to, granulocyte-colony stimulating factor (GCSF) and derivatives thereof, such as filgrastim and the long-acting GCSF PEG-filgrastim, or granulocyte-monocyte colony stimulating factor (GMCSF).

“Neutropenia” as used herein refers to an abnormally low concentration of neutrophils in the blood. “Reducing the incidence of neutropenia in a subject” refers to decreasing the number of neutropenia incidents in a subject receiving treatment and/or reducing the severity of neutropenic incidents in a subject. “Preventing neutropenia” refers to preventing or inhibiting the onset of neutropenia, e.g., as a result of prophylactic treatment with a granulopoiesis stimulating factor. Normal reference range for absolute neutrophil count (ANC) in adults is 1500 to 8000 cells per microliter (μl) of blood. Neutropenia can be categorized as follows: mild neutropenia (1000<=ANC<1500); moderate neutropenia (500<=ANC<1000); severe neutropenia (ANC<500). Hsieh et al., Ann. Intern. Med. 146:486-92, 2007.

The term “checkpoint inhibitor” as used herein refers to a molecule or therapeutic that blocks certain proteins made by some types of immune system cells, such as T cells, and some cancer cells. These proteins help keep immune responses in check and can keep T cells from killing cancer cells. Examples of checkpoint proteins found on T cells or cancer cells include PD-1, PD-L1, PD-L2, CD28, CTLA-4, B7-1, B7-2 (see National Cancer Institute Dictionary of Cancer Terms) as well as ICOS and BTLA.

“Programmed Death-1” (PD-1) refers to an immunoinhibitory receptor belonging to the CD28 family. PD-1 is expressed on previously activated T cells in vivo, and binds to two ligands, PD-L1 and PD-L2. The complete human PD-1 sequence can be found under GenBank Accession No. U64863.

“Programmed Death Ligand-1” (PD-L1) and PD-L2 are cell surface ligands for PD-1 that downregulate T cell activation and cytokine secretion upon binding to PD-1. The complete human PD-L1 sequence can be found under GenBank Accession No. Q9NZQ7.

The terms “specific binding” and “specifically binds” mean that the anti-CD30 antibody will react, in a highly selective manner, with its corresponding target, CD30, and not with the multitude of other antigens.

The term “monoclonal antibody” refers to an antibody that is derived from a single cell clone, including any eukaryotic or prokaryotic cell clone, or a phage clone, and not the method by which it is produced. Thus, the term “monoclonal antibody” as used herein is not limited to antibodies produced through hybridoma technology.

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In certain embodiments, the two sequences are the same length.

The term “substantially identical,” in the context of two nucleic acids or polypeptides, refers to two or more sequences or subsequences that have at least 70% or at least 75% identity; more typically at least 80% or at least 85% identity; and even more typically at least 90%, at least 95%, or at least 98% identity (for example, as determined using one of the methods set forth below).

The determination of percent identity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid encoding a protein of interest. BLAST protein searches can be performed with the XBLAST program, score=50, wordlength=3 to obtain amino acid sequences homologous to protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. 85:2444-8. Alternatively, protein sequence alignment may be carried out using the CLUSTAL W algorithm, as described by Higgins et al., 1996, Methods Enzymol. 266:383-402.

The abbreviation “MMAE” refers to monomethyl auristatin E.

The abbreviations “vc” and “val-cit” refer to the dipeptide valine-citrulline.

The abbreviation “PAB” refers to the self-immolative spacer:

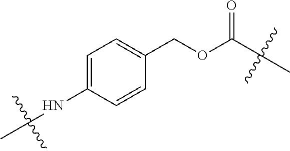 [SMILES: *Nc1ccc(COC(*)=O)cc1]

The abbreviation “MC” refers to the stretcher maleimidocaproyl:

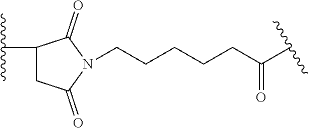 [SMILES: *C(=O)CCCCCN1C(=O)CC(*)C1=O]

cAC10-MC-vc-PAB-MMAE refers to a chimeric AC10 antibody conjugated to the drug MMAE through a MC-vc-PAB linker.

An anti-CD30 MC-vc-PAB-MMAE antibody-drug conjugate refers to an anti-CD30 antibody conjugated to the drug MMAE via a linker comprising the dipeptide valine citrulline and the self-immolative spacer PAB as shown in Formula (I) of U.S. Pat. No. 9,211,319.

The term “pharmaceutically acceptable” as used herein refers to those compounds, materials, compositions, and/or dosage forms that are, within the scope of sound medical judgment, suitable for contact with the tissues of human beings and animals without excessive toxicity, irritation, allergic response, or other problems or complications commensurate with a reasonable benefit/risk ratio. The term “pharmaceutically compatible ingredient” refers to a pharmaceutically acceptable diluent, adjuvant, excipient, or vehicle with which an antibody-drug conjugate is administered.

### Antibodies

Antibodies of the disclosure are preferably monoclonal, and may be multispecific, human, humanized or chimeric antibodies, single chain antibodies, Fab fragments, F(ab′) fragments, fragments produced by a Fab expression library, and binding fragments of any of the above. The term “antibody,” as used herein, refers to immunoglobulin molecules and immunologically active portions of immunoglobulin molecules, i.e., molecules that contain an antigen binding site that immunospecifically binds the antigen of interest. The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the antibodies are human antigen-binding antibody fragments of the present disclosure and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, may comprise the variable region(s) alone or in combination with the entirety or a portion of the following: hinge region, CH1, CH2, CH3 and CL domains. Also included in the disclosure are antigen-binding fragments also comprising any combination of variable region(s) with a hinge region, CH1, CH2, CH3 and CL domains. Preferably, the antibodies are human, murine (e.g., mouse and rat), donkey, sheep, rabbit, goat, guinea pig, camelid, horse, or chicken. As used herein, “human” antibodies include antibodies having the amino acid sequence of a human immunoglobulin and include antibodies isolated from human immunoglobulin libraries, from human B cells, or from animals transgenic for one or more human immunoglobulin, as described infra and, for example in U.S. Pat. No. 5,939,598 by Kucherlapati et al.

The antibodies of the present disclosure may be monospecific, bispecific, trispecific or of greater multi specificity. Multispecific antibodies may be specific for different epitopes of CD30 or may be specific for both CD30 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Antibodies of the present disclosure may be described or specified in terms of the particular CDRs they comprise. Additionally, antibodies of the present disclosure may also be described or specified in terms of their primary structures. Antibodies having at least 50%, at least 55%, at least 60%, at least 65%, at least 70%, at least 75%, at least 80%, at least 85%, at least 90%, at least 95% and most preferably at least 98% identity (as calculated using methods known in the art and described herein) to the variable regions described herein are also included in the present disclosure. Antibodies useful in the present methods disclosure may also be described or specified in terms of their binding affinity. Preferred binding affinities include those with a dissociation constant or Kd less than 5×10² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

The antibodies also include derivatives that are modified, i.e., by the covalent attachment of any type of molecule to the antibody such that covalent attachment does not prevent the antibody from binding to CD30 or from exerting a cytostatic or cytotoxic effect on Hodgkin's Disease cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies described herein may be generated by any suitable method known in the art.

Anti-CD30 Antibodies

Murine anti-CD30 mAbs known in the art have been generated by immunization of mice with Hodgkin's disease (HD) cell lines or purified CD30 antigen. AC10, originally termed 010 (Bowen et al., 1993, J. Immunol. 151:5896 5906), is distinct in that this anti-CD30 mAb that was prepared against a hum an NK-like cell line, YT (Bowen et al., 1993, J. Immunol. 151:5896 5906). Initially, the signaling activity of this mAb was evidenced by the down regulation of the cell surface expression of CD28 and CD45 molecules, the up regulation of cell surface CD25 expression and the induction of homotypic adhesion following binding of 010 to YT cells. Sequences of the AC10 antibody are set out in SEQ ID NO: 1-16 and Table A below. See also U.S. Pat. No. 7,090,843, incorporated herein by reference, which discloses a chimeric AC10 antibody.

Generally, antibodies of the disclosure immunospecifically bind CD30 and exert cytostatic and cytotoxic effects on malignant cells in Hodgkin's disease.

In various embodiments antibodies useful in the methods comprise one or more CDRs of AC10. The disclosure encompasses an antibody or derivative thereof comprising a heavy or light chain variable domain, said variable domain comprising (a) a set of three CDRs, in which said set of CDRs are from monoclonal antibody AC10, and (b) a set of four framework regions, in which said set of framework regions differs from the set of framework regions in monoclonal antibody AC10, and in which said antibody or derivative thereof immunospecifically binds CD30.

In various embodiments, the disclosure encompasses use of an antibody or derivative thereof comprising a heavy chain variable domain, said variable domain comprising (a) a set of three CDRs, in which said set of CDRs comprises SEQ ID NO:4, 6, or 8 and (b) a set of four framework regions, in which said set of framework regions differs from the set of framework regions in monoclonal antibody AC10, and in which said antibody or derivative thereof immunospecifically binds CD30.

In various embodiments, the disclosure encompasses use of an antibody or derivative thereof comprising a light chain variable domain, said variable domain comprising (a) a set of three CDRs, in which said set of CDRs comprises SEQ ID NO:12, 14 or 16, and (b) a set of four framework regions, in which said set of framework regions differs from the set of framework regions in monoclonal antibody AC10, and in which said antibody or derivative thereof immunospecifically binds CD30.

Antibodies having at least 50%, at least 55%, at least 60%, at least 65%, at least 70%, at least 75%, at least 80%, at least 85%, at least 90%, at least 95% and most preferably at least 98% identity (as calculated using methods known in the art and described herein) to the variable regions of AC10 are also included in the present disclosure, and preferably include the CDRs of AC10.

The disclosure further provides nucleic acids comprising a nucleotide sequence encoding a protein, including but not limited to, a protein of the disclosure and fragments thereof. Nucleic acids preferably encode one or more CDRs of antibodies that bind to CD30 and exert cytotoxic or cytostatic effects on HD cells. Exemplary nucleic acids comprise SEQ ID NO:3, SEQ ID NO:5, SEQ ID NO:7, SEQ ID NO:11, SEQ ID NO:13, or SEQ ID NO:15. Variable region nucleic acids of the disclosure comprise SEQ ID NO:1 or SEQ ID NO:9. (See Table A).

TABLE A

| | NUCLEOTIDE OR | |
|---|---|---|
| MOLECULE | AMINO ACID | SEQ ID NO |
| AC 10 Heavy Chain Variable Region | Nucleotide | 1 |
| AC 10 Heavy Chain Variable Region | Amino Acid | 2 |
| AC 10 Heavy Chain-CDR1 (H1) | Nucleotide | 3 |
| AC 10 Heavy Chain-CDR1 (H1) | Amino Acid | 4 |
| AC 10 Heavy Chain-CDR2 (H2) | Nucleotide | 5 |
| AC 10 Heavy Chain-CDR2 (H2) | Amino Acid | 6 |
| AC 10 Heavy Chain-CDR3 (H3) | Nucleotide | 7 |
| AC 10 Heavy Chain-CDR3 (H3) | Amino Acid | 8 |
| AC 10 Light Chain Variable Region | Nucleotide | 9 |
| AC 10 Light Chain Variable Region | Amino Acid | 10 |
| AC 10 Light Chain-CDR1 (L1) | Nucleotide | 11 |
| AC 10 Light Chain-CDR1 (L1) | Amino Acid | 12 |
| AC 10 Light Chain-CDR2 (L2) | Nucleotide | 13 |
| AC 10 Light Chain-CDR2 (L2) | Amino Acid | 14 |
| AC 10 Light Chain-CDR3 (L3) | Nucleotide | 15 |
| AC 10 Light Chain-CDR3 (L3) | Amino Acid | 16 |

In various embodiments, the antibody is an IgG antibody, e.g. an IgG1, IgG2, IgG3 or IgG4 antibody, preferably an IgG1 antibody.

In various embodiments, the anti-CD30 antibody of the anti-CD30 antibody drug conjugate comprises: i) a heavy chain CDR1 set out in SEQ ID NO: 4, a heavy chain CDR2 set out in SEQ ID NO: 6, a heavy chain CDR3 set out in SEQ ID NO: 8; and ii) a light chain CDR1 set out in SEQ ID NO: 12, a light chain CDR2 set out in SEQ ID NO: 14, and a light chain CDR3 set out in SEQ ID NO: 16.

In various embodiments, the anti-CD30 antibody of the anti-CD30 antibody drug conjugate comprises: i) an amino acid sequence at least 85%, 90% or 95% identical to a heavy chain variable region set out in SEQ ID NO: 2 and ii) an amino acid sequence at least 85%, 90%, or 95% identical to a light chain variable region set out in SEQ ID NO: 10. In various embodiments, the anti-CD30 antibody of the anti-CD30 antibody drug conjugate comprises: i) an amino acid heavy chain variable region set out in SEQ ID NO: 2 and ii) an amino acid light chain variable region set out in SEQ ID NO: 10.

In various embodiments, the anti-CD30 antibody (i) cross-competes with an antibody comprising a heavy chain CDR1 set out in SEQ ID NO: 4, a heavy chain CDR2 set out in SEQ ID NO: 6, a heavy chain CDR3 set out in SEQ ID NO: 8; and ii) a light chain CDR1 set out in SEQ ID NO: 12, a light chain CDR2 set out in SEQ ID NO: 14, and a light chain CDR3 set out in SEQ ID NO: 16. for binding to CD30; or (ii) binds to the same epitope as an antibody comprising a heavy chain CDR1 set out in SEQ ID NO: 4, a heavy chain CDR2 set out in SEQ ID NO: 6, a heavy chain CDR3 set out in SEQ ID NO: 8; and ii) a light chain CDR1 set out in SEQ ID NO: 12, a light chain CDR2 set out in SEQ ID NO: 14, and a light chain CDR3 set out in SEQ ID NO: 16.

Anti-PD-1 Antibodies

Human monoclonal antibodies that bind to PD-1 have been disclosed in U.S. Pat. Nos. 8,008,449, 6,808,710, 7,488,802, 8,168,757 and 8,354,509, and PCT Publication No. WO 2012/145493.

In one embodiment, the anti-PD-1 antibody is nivolumab. Nivolumab (also known as “OPDIVO®”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), blocking the down-regulation of antitumor T-cell functions (U.S. Pat. No. 8,008,449; Wang et al., 2014 Cancer Immunol Res. 2(9):846-56). In another embodiment, the anti-PD-1 antibody or fragment thereof cross-competes with nivolumab. In some embodiments, the anti-PD-1 antibody binds to the same epitope as nivolumab. In certain embodiments, the anti-PD-1 antibody has the same CDRs as nivolumab.

In one embodiment, the anti-PD-1 antibody is pembrolizumab. Pembrolizumab (“KEYTRUDA®”, lambrolizumab, MK-3475) is a humanized monoclonal IgG4 antibody directed against human cell surface receptor PD-1 (programmed death-1 or programmed cell death-1).

Pembrolizumab is described, for example, in U.S. Pat. No. 8,900,587. Pembrolizumab has been approved by the FDA for the treatment of relapsed or refractory melanoma and advanced NSCLC. In various embodiments, the anti-PD-1 antibody or antigen-binding portion thereof cross-competes with pembrolizumab. In various embodiments, the anti-PD-1 antibody binds to the same epitope as pembrolizumab. In various embodiments, the anti-PD-1 antibody has the same CDRs as pembrolizumab.

Additional anti-PD-1 antibodies contemplated for use herein include MED10680 (U.S. Pat. No. 8,609,089), BGB-A317 (U.S. Patent Publ. No. 2015/0079109),. INCSHR1210 (SHR-1210) (WO2015/085847), REGN-2810 (WO2015/112800) PDR001 (WO2015/112900), TSR-042 (ANB011) (WO2014/179664), and STI-1110 (WO2014/194302).

In various embodiments, the anti-PD-1 antibody or antigen-binding portion thereof is a chimeric, humanized or human monoclonal antibody or a portion thereof. In various embodiments, the antibody is a human or humanized antibody. Antibodies having an IgG1, IgG2, IgG3, or IgG4 isotype are contemplated.

In various embodiments, the anti-PD-1 antibody (i) cross-competes with nivolumab or pembrolizumab for binding to human PD-1; (ii) binds to the same epitope as nivolumab or pembrolizumab; (iii) is nivolumab; or (iv) is pembrolizumab.

### Antibody-Drug Conjugates

Contemplated herein is the use of antibody drug conjugates comprising an anti-CD30

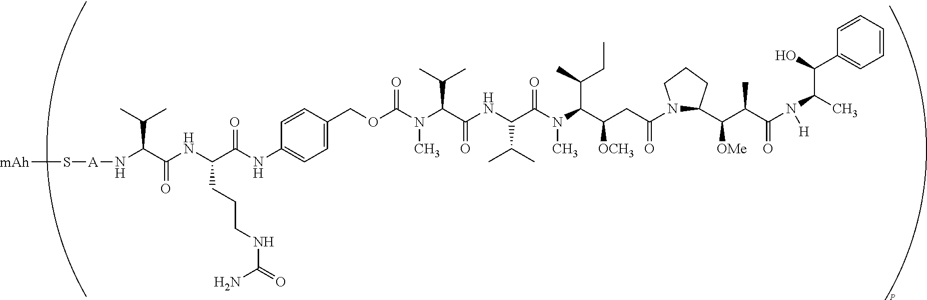

antibody, covalently linked to MMAE through a MC-vc-PAB linker. The antibody drug conjugates are delivered to the subject as a pharmaceutical composition. The anti-CD30 antibody drug conjugates are described in U.S. Pat. No. 9,211,319, herein incorporated by reference.

In various embodiments, the anti-CD30 antibody-drug conjugates of the present disclosure have the following formula: or a pharmaceutically acceptable salt thereof; wherein: mAb is an anti-CD30 antibody, S is a sulfur atom of the antibody, A—is a Stretcher unit, and p is from about 3 to about 5.

The drug loading is represented by p, the average number of drug molecules per antibody in a pharmaceutical composition. For example, if p is about 4, the average drug loading taking into account all of the antibody present in the pharmaceutical composition is about 4. P ranges from about 3 to about 5, more preferably from about 3.6 to about 4.4, even more preferably from about 3.8 to about 4.2. P can be about 3, about 4, or about 5. The average number of drugs per antibody in preparation of conjugation reactions may be characterized by conventional means such as mass spectroscopy, ELISA assay, and HPLC. The quantitative distribution of antibody-drug conjugates in terms of p may also be determined. In some instances, separation, purification, and characterization of homogeneous antibody-drug-conjugates where p is a certain value from antibody-drug-conjugates with other drug loadings may be achieved by means such as reverse phase HPLC or electrophoresis.

The Stretcher unit (A), is capable of linking an antibody unit to the valine-citrulline amino acid unit via a sulfhydryl group of the antibody. Sulfhydryl groups can be generated, for example, by reduction of the interchain disulfide bonds of an anti-CD30 antibody. For example, the Stretcher unit can be linked to the antibody via the sulfur atoms generated from reduction of the interchain disulfide bonds of the antibody. In some embodiments, the Stretcher units are linked to the antibody solely via the sulfur atoms generated from reduction of the interchain disulfide bonds of the antibody. In some embodiments, sulfhydryl groups can be generated by reaction of an amino group of a lysine moiety of an anti-CD30 antibody with 2-iminothiolane (Traut's reagent) or other sulfhydryl generating reagents. In certain embodiments, the anti-CD30 antibody is a recombinant antibody and is engineered to carry one or more lysines. In certain other embodiments, the recombinant anti-CD30 antibody is engineered to carry additional sulfhydryl groups, e.g., additional cysteines.

The synthesis and structure of MMAE is described in U.S. Pat. No. 6,884,869 incorporated by reference herein in its entirety and for all purposes. The synthesis and structure of exemplary Stretcher units and methods for making antibody drug conjugates are described in, for example, U.S. Publication Nos. 2006/0074008 and 2009/0010945 each of which is incorporated herein by reference in its entirety.

Representative Stretcher units are described within the square brackets of Formulas IIIa and IIIb of U.S. Pat. No. 9,211,319, and incorporated herein by reference.

In various embodiments, the anti-CD30 antibody drug conjugate comprises monomethyl auristatin E and a protease-cleavable linker. It is contemplated that the protease cleavable linker is comprises a thiolreactive spacer and a dipeptide. In various embodiments, the protease cleavable linker consists of a thiolreactive maleimidocaproyl spacer, a valine— citrulline dipeptide, and a p-amino-benzyloxycarbonyl spacer.

In a preferred embodiment, the anti-CD30 antibody drug conjugate is brentuximab vedotin, having the structure:

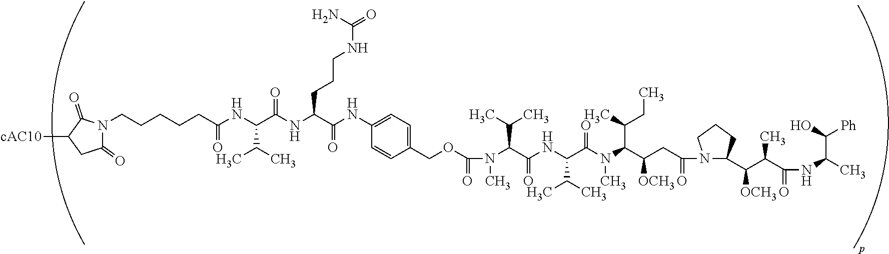

Brentuximab vedotin is a CD30-directed antibody-drug conjugate consisting of three components: (i) the chimeric IgG1 antibody cAC10, specific for human CD30, (ii) the microtubule disrupting agent MMAE, and (iii) a protease-cleavable linker that covalently attaches MMAE to cAC10. The drug to antibody ratio or drug loading is represented by “p” in the structure of brentuximab vedotin and ranges in integer values from 1 to 8. The average drug loading brentuximab vedotin in a pharmaceutical composition is about 4.

### Methods of Use

Provided herein are methods for administering an anti-CD30 antibody-drug conjugate in combination with additional cancer treating agents to treat hematologic cancers. Methods include treating a subject having hematologic cancer, e.g., a CD30-expressing hematologic cancer, by administering an anti-CD30 antibody drug conjugate, in combination with an anti-PD-1 antibody, and a chemotherapy regimen. In various embodiments, the chemotherapy regimen consists essentially of doxorubicin and dacarbazine.

Additional chemotherapeutic agents are disclosed in the following table and may be used alone or in combination with one or more additional chemotherapeutic agents, which in turn can also be administered in combination with an anti-CD30 antibody drug conjugate as described herein.

Chemotherapeutic Agents

| | |
|---|---|
| Alkylating agents | Natural products |
| Nitrogen mustards | Antimitotic drugs |
| mechlorethamine | Taxanes |
| cyclophosphamide | paclitaxel |
| ifosfamide | Vinca alkaloids |
| melphalan | vinblastine (VLB) |
| chlorambucil | vincristine |
| Nitrosoureas | vindesine |
| carmustine (BCNU) | vinorelbin |
| lomustine (CCNU) | Taxotere ® (docetaxel) |
| semustine (methyl-CCNU) | estramustine |
| Ethylenimine/Methyl-melamine | estramustine phosphate |
| thriethylenemelamine (TEM) | Epipodophylotoxins |
| triethylene thiophosphoramide | etoposide |
| (thiotepa) | teniposide |
| hexamethylmelamine | Antibiotics |
| (HMM, altretamine) | actimomycin D |
| Alkyl sulfonates | daunomycin (rubido-mycin) |
| busulfan | doxorubicin (adria-mycin) |
| Triazines | mitoxantrone |
| dacarbazine (DTIC) | idarubicin |
| Antimetabolites | epirubicin |
| Folic Acid analogs | valrubicin |
| methotrexate | bleomycin |
| Trimetrexate | splicamycin (mithramycin) |
| Pemetrexed | mitomycinC |
| (Multi-targeted antifolate) | dactinomycin |
| Pyrimidine analogs | aphidicolin |
| 5-fluorouracil | Enzymes |
| fluorodeoxyuridine | L-asparaginase |
| gemcitabine | L-arginase |
| cytosine arabinoside | Radiosensitizers |
| (AraC, cytarabine) | metronidazole |
| 5-azacytidine | misonidazole |
| 2,2′-difluorodeoxy-cytidine | desmethylmisonidazole |
| Purine analogs | pimonidazole |
| 6-mercaptopurine | etanidazole |
| 6-thioguanine | nimorazole |
| azathioprine | RSU 1069 |
| 2′-deoxycoformycin | EO9 |
| (pentostatin) | RB 6145 |
| erythrohydroxynonyl-adenine (EHNA) | SR4233 |
| fludarabine phosphate | nicotinamide |
| 2-chlorodeoxyadenosine | 5-bromodeozyuridine |
| (cladribine, 2-CdA) | 5-iododeoxyuridine |
| Type I Topoisomerase Inhibitors | bromodeoxycytidine |
| camptothecin | Miscellaneous agents |
| topotecan | bisphosphonates |
| irinotecan | RANKL inhibitor |
| Biological response modifiers | denosumab |
| G-CSF | Platinium coordination complexes |
| GM-CSF | cisplatin |
| Differentiation Agents | carboplatin |
| retinoic acid derivatives | oxaliplatin |
| Hormones and antagonists | nthracenedione |
| Adrenocorticosteroids/antagonists | mitoxantrone |
| calcitonin | Substituted urea |
| prednisone and equiv-alents | hydroxyurea |
| dexamethasone | Methylhydrazine derivatives |
| ainoglutethimide | N-methylhydrazine (MIH) |
| Progestins | procarbazine |
| hydroxyprogesterone caproate | Adrenocortical suppressant |
| medroxyprogesterone acetate | mitotane (o,p′-DDD) |
| megestrol acetate | ainoglutethimide |
| Estrogens | Cytokines |
| diethylstilbestrol | interferon (α, β, γ) |
| ethynyl estradiol/equivalents | interleukin-2 |
| Antiestrogen | Photosensitizers |
| tamoxifen | hematoporphyrin derivatives |
| Androgens | Photofrin ® |
| testosterone propionate | benzoporphyrin derivatives |
| fluoxymesterone/equivalents | Npe6 |
| Antiandrogens | tin etioporphyrin (SnET2) |
| flutamide | pheoboride-a |
| gonadotropin-releasing | bacteriochlorophyll-a |
| hormone analogs | naphthalocyanines |
| leuprolide | phthalocyanines |
| Nonsteroidal antiandrogens | zinc phthalocyanines |
| flutamide | Radiation |
| Histone Deacetylase Inhibitors | X-ray |
| Vorinostat | ultraviolet light |
| Romidepsin | gamma radiation |
| | visible light |
| | infrared radiation |
| | microwave radiation |

A hematologic or hematological cancer refers to a cancer that starts in blood forming tissue, or in cells of the immune system. A CD30-expressing hematologic cancer refers to a hematologic cancer that expresses the CD30 antigen. The CD30 antigen is expressed in large numbers on tumor cells of select lymphomas and leukemias. Hematological cancers such as classical Hodgkin lymphoma, non-Hodgkin lymphoma, anaplastic large-cell lymphoma, and cutaneous T-cell lymphoma (CTCL), are examples of hematologic cancers that can be treated by the present methods.

In various embodiments, the subject has a tumor comprising one or more cells that express CD30. In various embodiments, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the tumor cells express CD30.

It is also contemplated that the subject has a tumor expressing PD-1 or a ligand for PD-1, e.g., PD-L1 or PD-L2. Methods of measuring levels of PD-1, PD-L1 or PD-L2 known in the art are contemplated herein for determining levels of the molecules in a tumor cell. See, e.g., WO2017/210473.

In various embodiments, the PD-L1 expression level of a tumor is at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 75%, at least about 80%, at least about 85%, at least about 90%, at least about 95%, or about 100%.

In various embodiments, the anti-PD-1 antibody dose may be administered from at least about 0.1 mg/kg to at least about 10 mg/kg, from about 0.01 mg/kg to about 5 mg/kg, from about 1 mg/kg to about 5 mg/kg, from about 2 mg/kg to about 5 mg/kg, from about 1 mg/kg to about 3 mg/kg, or from about 7.5 mg/kg to about 12.5 mg/kg. In various embodiments, the anti-PD-1 antibody is given on a dose amount basis. In various embodiments, the dose of the anti-PD-1 antibody is from about 100-600 mg, from about 400-500 mg, from about 100-200 mg, from about 200-400 mg, or from about 100-300 mg. In various embodiments, the anti-PD-1 antibody is administered at a dose of about 60 mg, about 80 mg, about 100 mg, about 120 mg, about 130 mg, about 140 mg, about 160 mg, about 180 mg, about 200 mg, about 220 mg, about 240 mg, about 260 mg, about 280 mg, at least about 300 mg, about 320 mg, about 360 mg, about 400 mg, about 440 mg, about 480 mg, about 500 mg, about 550 mg, or about 600 mg.

In any of the aspects or embodiments herein, the methods herein provide for treating a subject who is newly diagnosed and has not previously been treated for a hematologic cancer, or a subject who has relapsed. In various embodiments, it is contemplated that the subject has classic Hodgkin Lymphoma (Stage IIA with bulky disease, Stage IIB, Stage III or Stage IV), including advanced classic Hodgkin Lymphoma (e.g., Stage III or Stage IV).

In various embodiments, the disclosure provides a method of treating a subject having newly diagnosed classical Hodgkin Lymphoma (HL) comprising administering an effective amount of a composition comprising brentuximab vedotin (A) and an anti-PD-1 antibody, in combination with a chemotherapy consisting essentially of doxorubicin, and dacarbazine (AD therapy), wherein the brentuximab vedotin is administered at 1.2 mg/kg, anti-PD-1 antibody is administered at 100-300 mg/dose, doxorubicin is administered at 25 mg/m², and dacarbazine is administered at 375 mg/m², and optionally, wherein the brentuximab vedotin is administered within 1 hour after administration of the AD therapy.

In various embodiments, the methods herein provide progression free survival (PFS) of the subject after therapy is maintained for greater than 1 year. In various embodiments, the progression free survival (PFS) of the subject after therapy is maintained for approximately 2 years. In certain embodiments, after four to six cycles of AN+AD therapy the subject has a Deauville score of 3 or less, or 2 or less. In certain embodiments, after two cycles of therapy [i.e., four administrations] the subject has a Deauville score of 1 or 2.

In various embodiments, if the anti-CD30 antibody drug conjugate is administered at 1.2 mg/kg with combination therapy, e.g., N+AD, the combination therapy is administered every two weeks. For example, the combination therapy is administered on days 1 and 15 of a 28 day cycle.

In various embodiments, the anti-CD30 antibody drug conjugate +N+AD combination therapy is administered for no more than six cycles, for examples from 4 to 6 cycles, or for 4, 5 or 6 cycles.

It is contemplated that the therapy is administered until a PET scan determines there is no tumor or progression of tumor. If after the end of treatment, e.g., 4 to 6 cycles, the PET scan still shows some tumor, the treating physician may repeat the course of treatment as necessary until the PET scan is negative or shows slowed or no tumor progression. The repeat of cycles may begin after no break, or after 1, 2, 3, 4, 5, 6 or more weeks after the initial treatment with AN+AD therapy.

In various embodiments, anti-CD30 antibody drug conjugate, e.g., brentuximab vedotin, therapy is administered by intravenous infusion over the course of about 30 minutes. In various embodiments, anti-PD-1 antibody is administered by intravenous infusion over the course of about 60 minutes.

In various embodiments, the hematologic cancer is selected from the group consisting of classical Hodgkin Lymphoma, non-Hodgkin Lymphoma, cutaneous T-cell lymphoma (CTCL), and anaplastic large cell lymphoma (ALCL).

In various embodiments, the hematologic cancer is classical Hodgkin Lymphoma. In various embodiments, the hematologic cancer is a stage III or IV classical Hodgkin Lymphoma. In various embodiments, the hematologic cancer of the subject has not been treated.

In various embodiments, the anaplastic large cell lymphoma (ALCL) is a systemic anaplastic large cell lymphoma (sALCL).

In various embodiments, the cutaneous T-cell lymphoma (CTCL) is a mycosis fungoides (MF). In various embodiments, the mycosis fungoides (MF) is a CD30-positive mycosis fungoides (MF). In various embodiments, the cutaneous T-cell lymphoma (CTCL) is a primary cutaneous anaplastic large cell lymphoma (pcALCL).

In various embodiments, the subject has received prior systemic therapy or prior radiation

Effects of the treatment described herein can be measured using various biomarkers, progression of tumor size or other symptoms in a subject, including reducing serum thymus and activation-regulated chemokine (TARC) levels in the subject, increasing the level of a pro-inflammatory cytokine, e.g., Interleukin-18 (IL-18) and/or Interferon-γ, in the subject, increasing the level of a T cell chemokine, e.g., IP10, in a subject, activate T cell activity (e.g., increase the number of T cells, CD4+ T cells, regulatory T cells (Tregs)), and slowing or reducing tumor cell growth. In various embodiments, tumor cell growth is slowed or reduced by at least about 10%, by at least about 20%, by at least about 30%, by at least about 40%, by at least about 50%, by at least about 60%, by at least about 70%, or by at least about 80%, by at least about 90%, at least about 95%, or at least about 100% relative to untreated subjects or subject receiving a different therapeutic regimen.

Peripheral Neuropathy

Neuropathy, or peripheral neuropathy, refers to damage to the nerves outside of the brain and spinal cord (peripheral nerves). Peripheral neuropathy develops as a result of damage to the peripheral nervous system during treatment with anti-CD30 antibody drug conjugate. Symptoms include numbness or tingling, pricking sensations (paresthesia), and muscle weakness. Motor nerve damage is most commonly associated with muscle weakness.

The method contemplates dose modification of anti-CD30 drug conjugate if neuropathy appears in a subject. It is contemplated that the neuropathy is peripheral neuropathy. Provided herein is a method for treating a subject that has exhibited Grade 2 or greater peripheral neuropathy after starting administration of anti-CD30 antibody drug conjugate, combination therapy as described herein, e.g., wherein brentuximab vedotin is administered at a dose of 1.2 mg/kg or more, comprising administering the anti-CD30 antibody drug conjugate at a dose of 0.9 mg/kg. In various embodiments, when the subject exhibits Grade 3 neuropathy, the administration of the anti-CD30 antibody drug conjugate, e.g., brentuximab vedotin, is withheld until peripheral neuropathy decreases to Grade 2 or lower and then 0.9 mg/kg of the anti-CD30 antibody drug conjugate is administered. In some embodiments, the reduced dose of 0.9 mg/kg is given up to a maximum dose of 90 mg every 2 weeks. In various embodiments, when the subject exhibits Grade 4 neuropathy, the administration of the anti-CD30 antibody drug conjugate, e.g., brentuximab vedotin, is withheld until peripheral neuropathy decreases to Grade 2 or lower and then 0.9 mg/kg of the anti-CD30 antibody drug conjugate is administered.

In various embodiments, when the subject exhibits Grade 3 neuropathy, the administration of anti-CD30 antibody drug conjugate is reduced, e.g., to 0.9 mg/kg, until peripheral neuropathy decreases to Grade 2 or less and then 0.9 mg/kg anti-CD30 antibody drug conjugate is administered or maintained. In various embodiments, when the peripheral neuropathy is a Grade 2, the reduced dose of 0.9 mg/kg is given up to a maximum dose of 90 mg every 2 weeks.

Methods for measuring neuropathy are known in the art and utilized by the treating physician to monitor and diagnose neuropathy in a subject receiving anti-CD30 antibody drug conjugate therapy. For example, the National Cancer Information Center-Common Toxicity Criteria (NCIC-CCT) describes Grade 1 PN as characterized by mild paresthesias and/or loss of deep tendon flexion; Grade 2 PN is characterized by mile or moderate objective sensory loss and/or moderate paresthesias; Grade 3 PN is characterized by sensory loss and/or paresthesias that interferes with function. Grade 4 PN is characterized by paralysis.

In various embodiments, the dose of anti-CD30 antibody drug conjugate is delayed by one week, or two weeks, if peripheral neuropathy appears, and therapy is continued when the neuropathy is resolved or determined to be Grade 2 or less or Grade 1 or less.

Neutropenia

Neutropenia is a common side effect of chemotherapy regimens and results from depletion of neutrophils in the blood of patients receiving chemotherapeutic treatment. Neutropenia is also observed in treatment with brentuximab vedotin. Neutropenia is commonly diagnosed based on levels of neutrophils in the blood. For example, Grade 3 neutropenia refers to an absolute blood neutrophil count [ANC]<1.0×10⁹/I); Grade 4 neutropenia refers to absolute blood neutrophil count [ANC]<0.5×10⁹/I), Febrile neutropenia refers to neutropenia with fever, the subject having a single oral temperature 38.3° C. or 38.0° C. for >1 h, with grade 3/4 neutropenia.

It is contemplated herein that subjects receiving an anti-CD30 antibody drug conjugate, e.g., brentuximab vedotin, or anti-CD30 antibody drug conjugate in combination with anti-PD-1 antibody and chemotherapy, such as AD combination therapy, receive granulopoiesis stimulating factors prophylactically beginning with cycle 1 of the administration of the anti-CD30 antibody drug conjugate, e.g., as primary prophylaxis. Exemplary granulopoiesis stimulating factors include granulocyte colony stimulating factor (GCSF), derivatives of GCSF, or granulocyte monocyte colony stimulating factor (GMCSF). Commercially available GCSF contemplated for use herein are filgrastim (NEUPOGEN®) and pegfilgrastim (NEULASTA®). Commercially available GMCSF is available as sargramostim (LEUKINE®).

Provided herein is a method for treating a hematologic cancer in a subject comprising administering an anti-CD30 antibody drug conjugate combination therapy as described herein (AN+AD) and prophylactically administering a granulopoiesis stimulating factor beginning with cycle 1 of the administration of the anti-CD30 antibody drug conjugate, wherein the granulopoiesis stimulating factor is administered within 1 day to within 7 days after beginning with cycle 1 of the administration of the anti-CD30 antibody drug conjugate. In further embodiments, the granulopoiesis stimulating factor is administered from within 1 day or 2 days to within 5 days after beginning with cycle 1 of the administration of the anti-CD30 antibody drug conjugate. In various embodiments, the granulopoiesis stimulating factor is administered about 24 hours to about 36 hours after each administration of anti-CD30 antibody drug conjugate, optionally anti-CD30 antibody drug conjugate in combination with a chemotherapy regimen described herein. In various embodiments, the granulopoiesis stimulating factor is administered 24 hours to 36 hours after each administration of, i.e., after each dose of, anti-CD30 antibody drug conjugate.

Also contemplated is a method for reducing the incidence of neutropenia and/or febrile neutropenia in a subject receiving treatment with an anti-CD30 antibody drug conjugate comprising administering to the subject a granulopoiesis stimulating factor, wherein the stimulating factor is administered from 1 day to 7 days beginning with cycle 1 of the administration of the anti-CD30 antibody drug conjugate, optionally from 1 day or 2 days to 5 days after beginning with cycle 1 of the administration of the anti-CD30 antibody drug conjugate. In various embodiments, the subject has febrile neutropenia and is 60 years old or older. In various embodiments, the granulopoiesis stimulating factor is administered about 24 hours to about 36 hours after each administration of anti-CD30 antibody drug conjugate, optionally anti-CD30 antibody drug conjugate in combination with a chemotherapy regimen described herein. In various embodiments, the granulopoiesis stimulating factor is administered 24 hours to 36 hours after each administration of anti-CD30 antibody drug conjugate.

Further contemplated is a method wherein the granulopoiesis stimulating factor is administered from 1 day to 7 days after a second, or subsequent, administration of anti-CD30 antibody drug conjugate. In certain embodiments, the granulopoiesis stimulating factor is administered from 1 day or 2 days to 5 days after the second or subsequent administration of anti-CD30 antibody drug conjugate. In various embodiments, the granulopoiesis stimulating factor is administered about 24 hours to about 36 hours after each administration of anti-CD30 antibody drug conjugate, optionally anti-CD30 antibody drug conjugate in combination with a chemotherapy regimen described herein. In various embodiments, the granulopoiesis stimulating factor is administered 24 hours to 36 hours after each administration of anti-CD30 antibody drug conjugate.

In various embodiments, the subject that has not received anti-CD30 antibody drug conjugate therapy previously. In various embodiments, the subject has not experienced treatment-emergent Grade 3-4 neutropenia after anti-CD30 antibody drug conjugate administration.

It is contemplated that the granulopoiesis stimulating factor is granulocyte colony stimulating factor (GCSF). It is contemplated that the GCSF is a long-acting GCSF or not a long acting GCSF.

In various embodiments, when the stimulating factor is not long-acting GCSF, e.g. filgrastim, it can be administered starting from 1 to 7 days, from 1 to 5 days, or 1 to 3 days after beginning with cycle 1 of the administration of the anti-CD30 antibody drug conjugate or AN+AD therapy, e.g. in daily doses. In certain embodiments, the GCSF is administered on day 2, 3, 4, 5, 6 and/or 7 after anti-CD30 antibody drug conjugate or AN+AD therapy. In various embodiments, the filgrastim is administered at a dose of 5 ug/kg/day to 10 ug/kg/day for the duration of at least 3, 4, 5, 6, 7, 8, 9, 10, 11, 12, 13 or 14 days.

Pegfilgrastim is a long-lasting, PEGylated form of filgrastim that has a longer half-life in vivo. In various embodiments, pegfilgrastim is administered at 6 mg/dose from 1 day to 5 days after anti-CD30 antibody drug conjugate treatment, or optionally after AN+AD therapy. In certain embodiments, the GCSF is administered in a single dose, or a multiple dose on the same day, on day 2, day 3, day 4 or day 5 after anti-CD30 antibody drug conjugate or AN+AD therapy. In various embodiments, the GCSF is administered about 24 hours to about 36 hours after each administration of anti-CD30 antibody drug conjugate, optionally anti-CD30 antibody drug conjugate in combination with a chemotherapy regimen described herein. In various embodiments, the G-CSF is administered 24 hours to 36 hours after each administration of anti-CD30 antibody drug conjugate.

In various embodiments, the granulopoiesis stimulating factor is administered intravenously or subcutaneously. It is contemplated that the granulopoiesis stimulating factor is given in a single dose or multiple doses, e.g., in multiple daily doses.

It is contemplated that a subject receiving a granulopoiesis stimulating factor and anti-CD30 antibody drug conjugate may also be administered an antibiotic to address issues of febrile neutropenia and/or infection. Exemplary antibiotics contemplated include those known in the art, such as cephalosporin, sulfamethoxazole-trimethoprim, ACYCOLOVIR®, FLUCANOZOLE®, or INTRACONAZOLE®.

In various embodiments, if the subject is receiving 1.2 mg/kg of anti-CD30 antibody drug conjugate every two weeks, the dose may be reduced to 0.9 mg/kg to improve neutropenia, e.g., Grade 4 neutropenia.

### Formulations

Various delivery systems can be used to administer antibodies or antibody-drug conjugates contemplated herein. In certain embodiments, administration of the antibody-drug conjugate compound is by intravenous infusion. In some embodiments, administration is by a 30 minute, 1 hour or two hour intravenous infusion. In various embodiments, administration of the antibody compound is by intravenous infusion. In various embodiments, administration is by a 30 minute, 1 hour or two hour intravenous infusion.

The antibody and/or antibody-drug conjugate compound can be administered as a pharmaceutical composition comprising one or more pharmaceutically compatible ingredients. For example, the pharmaceutical composition typically includes one or more pharmaceutically acceptable carriers, for example, water-based carriers (e.g., sterile liquids). Water is a more typical carrier when the pharmaceutical composition is administered intravenously.

The composition, if desired, can also contain, for example, saline salts, buffers, salts, nonionic detergents, and/or sugars. Examples of suitable pharmaceutical carriers are described in “Remington's Pharmaceutical Sciences” by E. W. Martin. The formulations correspond to the mode of administration.

The present disclosure provides, for example, pharmaceutical compositions comprising a therapeutically effective amount of the antibody-drug conjugate, a buffering agent, optionally a cryoprotectant, optionally a bulking agent, optionally a salt, and optionally a surfactant. Additional agents can be added to the composition. A single agent can serve multiple functions. For example, a sugar, such as trehalose, can act as both a cryoprotectant and a bulking agent. Any suitable pharmaceutically acceptable buffering agents, surfactants, cyroprotectants and bulking agents can be used in accordance with the present disclosure.

In various embodiments, the antibody drug conjugate formulations including drug conjugate formulations have undergone lyophilization, or other methods of protein preservation, as well as antibody drug formulations that have not undergone lyophilization.

In some embodiments, the antibody drug conjugate formulation comprises (i) about 1-25 mg/ml, about 3 to about 10 mg/ml of an antibody-drug conjugate, or about 5 mg/ml (e.g., an antibody-drug conjugate of formula I or a pharmaceutically acceptable salt thereof), (ii) about 5-50 mM, preferably about 10 mM to about 25 mM of a buffer selected from a citrate, phosphate, or histidine buffer or combinations thereof, preferably sodium citrate, potassium phosphate, histidine, histidine hydrochloride, or combinations thereof, (iii) about 3% to about 10% sucrose or trehalose or combinations thereof, (iv) optionally about 0.05 to 2 mg/ml of a surfactant selected from polysorbate 20 or polysorbate 80 or combinations thereof; and (v) water, wherein the pH of the composition is from about 5.3 to about 7, preferably about 6.6.

In some embodiments, an antibody drug conjugate formulation will comprise about 1-25 mg/ml, about 3 to about 10 mg/ml, preferably about 5 mg/ml of an antibody-drug conjugate, (ii) about 10 mM to about 25 mM of a buffer selected from sodium citrate, potassium phosphate, histidine, histidine hydrochloride or combinations thereof, (iii) about 3% to about 7% trehalose or sucrose or combinations thereof, optionally (iv) about 0.05 to about 1 mg/ml of a surfactant selected from polysorbate 20 or polysorbate 80, and (v) water, wherein the pH of the composition is from about 5.3 to about 7, preferably about 6.6.

In some embodiments, an antibody drug conjugate formulation will comprise about 5 mg/ml of an antibody-drug conjugate, (ii) about 10 mM to about 25 mM of a buffer selected from sodium citrate, potassium phosphate, histidine, histidine hydrochloride or combinations thereof, (iii) about 3% to about 7% trehalose, optionally (iv) about 0.05 to about 1 mg/ml of a surfactant selected from polysorbate 20 or polysorbate 80, and (v) water, wherein the pH of the composition is from about 5.3 to about 7, preferably about 6.6.

In various embodiments, the anti-PD-1 antibody, e.g., nivolumab, is in a solution of about 40 mg/4 mL, 100 mg/10 mL, or 240 mg/24 mL. Optionally, the anti-PD-1 antibody is diluted in 0.9% Sodium Chloride, USP or 5% Dextrose.

In various embodiments, the anti-PD-1 antibody, e.g., pembrolizumab, is in a 50 mg lyophilized powder, or 100 mg/4 mL (25 mg/mL) in solution. In various embodiments, the preparation is in a solution containing 0.9% Sodium Chloride Injection, USP or 5% Dextrose Injection, USP. The final concentration of the diluted solution can be between 1 mg/mL to 10 mg/mL.

Any of the formulations described above can be stored in a liquid or frozen form and can be optionally subjected to a preservation process. In some embodiments, the formulations described above are lyophilized, i.e., they are subjected to lyophilization. In some embodiments, the formulations described above are subjected to a preservation process, for example, lyophilization, and are subsequently reconstituted with a suitable liquid, for example, water. By lyophilized it is meant that the composition has been freeze-dried under a vacuum. Lyophilization typically is accomplished by freezing a particular formulation such that the solutes are separated from the solvent(s). The solvent is then removed by sublimation (i.e., primary drying) and next by desorption (i.e., secondary drying).

The formulations of the present disclosure can be used with the methods described herein or with other methods for treating disease. The antibody drug conjugate formulations may be further diluted before administration to a subject. In some embodiments, the formulations will be diluted with saline and held in IV bags or syringes before administration to a subject. Accordingly, in some embodiments, the methods for treating a hematologic cancer in a subject will comprise administering to a subject in need thereof a weekly dose of a pharmaceutical composition comprising antibody-drug conjugates having formula wherein the administered dose of antibody-drug conjugates is from about 1.2 mg/kg of the subject's body weight to 0.9 mg/kg of the subject's body weight and the pharmaceutical composition is administered for at least two weeks and wherein the antibody drug conjugates, prior to administration to a subject, were present in a formulation comprising (i) about 1-25 mg/ml, preferably about 3 to about 10 mg/ml of the antibody-drug conjugate (ii) about 5-50 mM, preferably about 10 mM to about 25 mM of a buffer selected from sodium citrate, potassium phosphate, histidine, histidine hydrochloride, or combinations thereof, (iii) about 3% to about 10% sucrose or trehalose or combinations thereof, (iv) optionally about 0.05 to 2 mg/ml of a surfactant selected from polysorbate 20 or polysorbate 80 or combinations thereof; and (v) water, wherein the pH of the composition is from about 5.3 to about 7, preferably about 6.6.

Formulations of chemotherapeutics contemplated for use herein, including doxorubicin, and dacarbazine are provided as typically used in the treatment of cancers. For example, doxorubicin and dacarbazine are commercially available and approved by the United States FDA and other regulatory agencies for use in treating patients with multiple types of cancer.

The present disclosure also provides kits for the treatment of a hematologic cancer. The kit can comprise (a) a container containing the antibody-drug conjugate and optionally, containers comprising one or more of anti-PD-1 antibody, doxorubicin, or dacarbazine. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit.

### EXAMPLES

### Example 1

Described herein is an open-label, multicenter, phase 2 trial to assess efficacy of brentuximab vedotin, anti-PD-1 antibody (nivolumab), doxorubicin, and dacarbazine (AN+AD) as frontline therapy in patients with previously untreated stage III/IV classical Hodgkin lymphoma (cHL).

The combination of brentuximab vedotin and nivolumab appears to be active and well tolerated in cHL. In one trial in 62 subjects in the first salvage setting, the combination produced a 61% CR rate (Herrera et al., 2018 Blood 131(11): 1183-94) and subjects were able to undergo subsequent stem cell transplant. In another trial of 19 subjects with relapsed/refractory cHL who had received a median of 3 prior therapies, the combination produced a 50% CR rate (Diefenbach et al., 2017, Hematol Oncol 35(Suppl 2): 84-5). In another trial in 11 previously untreated subjects with cHL over 60 years-old and ineligible for declining conventional combination chemotherapy, the combination of brentuximab vedotin and nivolumab produced a 55% CR rate (Friedberg et al., 2018 HemaSphere 2(S3): T027 (0153)). No new safety signals were identified and the combination was considered to be well tolerated.

The combination of nivolumab has also been shown to be well tolerated when combined with doxorubicin, vinblastine, and dacarbazine (N+AVD). Ramchandren and colleagues observed a 67% CR rate with the multi-agent combination in 51 subjects with newly-diagnosed advanced stage cHL (Ramchandren et al., 2019 J Clin Oncol 37(23): 1997-2007). Neutropenia was reported for 55% of subjects and treatment-related febrile neutropenia was reported for 10% subjects.

### Materials and Methods

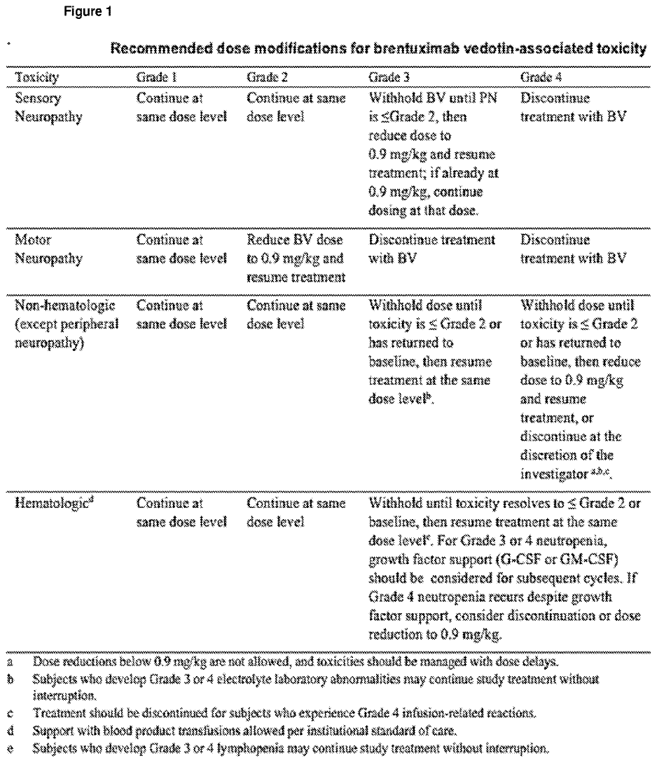

TRIAL DESIGN: Patients receive AN+AD (brentuximab vedotin 1.2 mg/kg, nivolumab 240 mg, doxorubicin 25 mg/m², dacarbazine 375 mg/m²) intravenously on days 1 and 15 of each 28-day cycle for up to 6 cycles. Brentuximab vedotin is administered intravenously over a period of approximately over 30 minutes. Nivolumab is administered intravenously over an approximately 60-minute infusion, and is administered at least 30 minutes after completion of brentuximab vedotin administration. Doxorubicin and dacarbazine are administered per institutional standards. Dose reductions/modifications of brentuximab vedotin are described in FIG. 1.

PATIENTS: Patients (12 years of age) with histologically confirmed (Ann Arbor stage IIA with bulky disease/IIB/III/IV) classical Hodgkin lymphoma according to the World Health Organisation Classification, not previously treated with systemic chemotherapy/radiotherapy, are eligible. Patients are required to have an Eastern Cooperative Oncology Group performance status and satisfactory absolute neutrophil and platelet counts, hemoglobin levels, and liver and kidney function marker levels (except for patients with involvement of the marrow or liver or Gilbert syndrome). Patients with nodular lymphocyte-predominant Hodgkin lymphoma are ineligible, as are those previously treated with a checkpoint inhibitor or T-cell costimulatory therapy, previously treated with brentuximab vedotin, with peripheral sensory/motor neuropathy, a positive pregnancy test, known cerebral/meningeal disease, any evidence of residual disease from another malignancy or diagnosis of another malignancy within 3 years before the first dose, interstitial lung disease, Grade 3 or higher pulmonary disease, idiopathic interstitial pneumonia or diffusing capacity of the lung for carbon monoxide, subjects with Child-Pugh B or C hepatic impairment, or clinically relevant cardiovascular conditions.

Exemplary checkpoint inhibitors interfere with activity of one or more of the following, PD-1, PD-L1, PD-L2, CD28, ICOS, CTLA-4 and BTLA. In addition to the anti-PD-1 antibodies nivolumab, pembrolizumab and others described herein, checkpoint inhibitors include ipilimumab (YERVOY®), which binds to and inhibits CTLA-4.

ENDPOINTS: The primary endpoint is complete response rate (CR). Secondary endpoints include assessment of safety and tolerability of treatment as well as overall response rate, duration of response, duration of complete response, event free survival, progression free survival and overall survival.

Complete Response Rate (CR) at end of therapy (EOT) is defined as the proportion of subjects with CR at EOT, according to the Lymphoma Response to Immunomodulatory Therapy Criteria (LYRIC) (Cheson et al., 2016 Blood 128(21): 2489-96), in subjects with previously untreated advanced cHL. Subjects who do not have a post-baseline assessment will be scored as non-responders for calculating the CR rate at EOT.

Objective Response Rate (ORR) is defined as the proportion of subjects with CR or partial response (PR) at EOT according to the Lymphoma Response to Immunomodulatory Therapy Criteria (LYRIC) (Cheson 2016, supra) in subjects with previously untreated advanced cHL.

Duration of response (DOR) is defined as the time from the first documentation of objective tumor response (CR or PR) to the first documentation of tumor progression (per the Lymphoma Response to Immunomodulatory Therapy Criteria (LYRIC) (Cheson 2016, supra) or death, whichever comes first. Subjects without progression or death will be censored; details will be provided in the statistical analysis plan (SAP). Duration of response will only be calculated for the subgroup of subjects achieving a CR or PR.

Duration of complete response (DOCR) is defined as the time from start of the first documentation of complete tumor response (CR) to the first documentation of tumor progression (per the Lymphoma Response to Immunomodulatory Therapy Criteria (LYRIC) (Cheson 2016, supra) or death, whichever comes first. DOCR will only be calculated for the subgroup of subjects achieving CR. Censoring will be in a manner similar to DOR.

Event-free survival (EFS) is defined as the time from the date of randomization to the first documentation of objective tumor progression, death due to any cause, or receipt of subsequent anticancer therapy to treat residual or progressive disease, whichever occurs first.

Progression-free Survival PFS is defined as the time from start of study treatment to first documentation of objective tumor progression or death.

Overall survival is defined as the time from start of study treatment to date of death due to any cause. In the absence of confirmation of death, survival time will be censored at the last date the subject is known to be alive.

ASSESSMENTS: The determination of antitumor activity is based on objective response assessments made according to Lugano Classification Revised Staging System for malignant lymphoma (Cheson et al., 2014 J. Clin Oncol 32(27): 3059-68) with the incorporation of Lymphoma Response to Immunomodulatory Therapy Criteria (LYRIC) (Cheson 2016, supra). ECOG performance status is testedin cycles 2 to 6.

Staging is performed by CT of diagnostic quality and PET scan, with disease involvement determined by focal fluorodeoxyglucose (FDG) uptake in nodal and extranodal (including spleen, liver, bone marrow, and thyroid) sites that is consistent with lymphoma, according to the pattern of uptake and/or CT characteristics. A CT of diagnostic quality may be combined with the PET scan when both are required per protocol. Up to 6 of the largest nodes, nodal masses, or other involved lesions that are measurable in 2 diameters should be identified as target lesions at baseline.

Progressive metabolic disease (PmD), no metabolic response (NmR), partial metabolic response (PmR), or complete metabolic response (CmR) is determined using PET-based response at each assessment. If only CT-based assessment is performed, response is categorized as progressive disease (PD), stable disease (SD), partial response (PR), or complete response (CR). If clinical progression is determined by the investigator, radiographic staging should also be performed to determine response assessment per Lugano classification criteria. The PET scan metabolic uptake will be graded using the Deauville 5-point scale (Barrington et al., 2010 Eur J Nucl Med Mol Imaging 37(10): 1824-33; Biggi et al., 2013 J Nucl Med 54(5): 683-90) with a score of ≤3 considered to represent a complete metabolic response. Both PET and CT scanning are used until disease is PET negative; responses will then be followed by CT scan of diagnostic quality only.

Treatment with checkpoint inhibitors, such as nivolumab, can result in false positive PET imaging. LYRIC criteria recommends repeat PET imaging and/or biopsy to further evaluate PET-positive (D4 or D5) lesions identified at the EOT response assessment.

Safety is evaluated by the incidence of adverse events, using the Medical Dictionary for Regulatory Activities (MedDRA; v19.0), and National Cancer Institute Common Terminology Criteria for Adverse Events v4.03, and by changes in vital signs, and clinical laboratory results.

Biomarker assessments in tumor tissue may include, but are not limited to, measurements of CD30, PD-L1, characterization of the tumor microenvironment (e.g., levels of T cell, NK cells, monocytes and macrophages, other tumor associated cells), tumor subtyping, profiling of somatic mutations or alterations in genes or RNA commonly altered in cancer, and drug effects. Assays may include, but are not limited to, immunohistochemistry and next generation sequencing of RNA and DNA.

LAB ASSESSMENTS: The following laboratory assessment(s) are performed by local laboratories at scheduled timepoints. The chemistry panel includes the following tests: albumin, alkaline phosphatase, ALT, AST, blood urea nitrogen, calcium, creatinine, chloride, glucose, lactate dehydrogenase (LDH), phosphorus, potassium, sodium, total bilirubin, and uric acid. For Part B, the chemistry panel should also include amylase and lipase; TSH, free T3, and free T4 is also be tested at Cycle 1, Cycle 3, and EOT.

The complete blood count (CBC) with differential includes the following tests: white blood cell count with five-part differential (neutrophils, lymphocytes, monocytes, eosinophils, and basophils), platelet count, hemoglobin, and hematocrit.

The estimated glomelular filtration rate (GFR) is calculated using the MDRD equation as applicable, with serum creatinine (Scr) reported in mg/dL.

STATISTICAL ANALYSIS: ORR at EOT and the exact 2-sided 95% Cls using the Clopper-Pearson method (Clopper 1934 Biometrika 26(4): 404-13) is calculated. Secondary endpoints of DOR, EFS, PFS, and OS are time-to-event endpoints and will be analyzed using Kaplan-Meier methodology.

Numerous modifications and variations of the disclosure as set forth in the above illustrative examples are expected to occur to those skilled in the art. Consequently only such limitations as appear in the appended claims should be placed on the disclosure.

What is claimed:

1. A method for treating a hematologic cancer in a subject comprising administering a therapy comprising an anti-CD30 antibody drug conjugate and an anti-PD-1 antibody, doxorubicin and dacarbazine.

2. The method of claim 1, wherein the anti-PD-1 antibody is administered at least 30 minutes after each administration of anti-CD30 antibody drug conjugate.

3. The method of claim 1 or 2, wherein the anti-PD-1 antibody is administered by intravenous infusion for a duration of approximately 60 minutes.

4. The method of any one of the preceding claims, wherein the anti-PD-1 antibody is administered to a subject that has not received anti-CD30 antibody drug conjugate therapy previously.

5. The method of any one of the preceding claims, wherein the anti-CD30 antibody drug conjugate is administered to a subject that has not received anti-CD30 antibody drug conjugate therapy previously.

6. The method of any one of the preceding claims, wherein the anti-CD30 antibody drug conjugate and anti-PD-1 antibody are administered every 2 weeks.

7. The method of claim 1 wherein the anti-PD-1 antibody is administered beginning with cycle 1 of the administration of anti-CD30 antibody drug conjugate.

8. The method of anyone of the preceding claims, wherein the anti-CD30 antibody drug conjugate and anti-PD-1 antibody are administered on day 1 and day 15 of a 28-day cycle.

9. The method of any one of the preceding claims, wherein the anti-CD30 antibody drug conjugate and anti-PD-1 antibody are administered for no more than six cycles.

10. The method of any one of the preceding claims, wherein the anti-CD30 antibody drug conjugate and anti-PD-1 antibody are administered for four to six cycles.

11. The method of any one of the preceding claims further comprising administering a chemotherapy consisting essentially of doxorubicin and dacarbazine (AD) as a combination therapy.

12. The method of any one of the preceding claims, wherein the anti-CD30 antibody of the anti-CD30 antibody drug conjugate comprises
i) a heavy chain CDR1 set out in SEQ ID NO: 4, a heavy chain CDR2 set out in SEQ ID NO: 6, a heavy chain CDR3 set out in SEQ ID NO: 8; and
ii) a light chain CDR1 set out in SEQ ID NO: 12, a light chain CDR2 set out in SEQ ID NO: 14, and a light chain CDR13 set out in SEQ ID NO: 16.

13. The method of any one of the preceding claims, wherein the anti-CD30 antibody of the anti-CD30 antibody drug conjugate comprises
i) an amino acid sequence at least 85% identical to a heavy chain variable region set out in SEQ ID NO: 2 and
ii) an amino acid sequence at least 85% identical to a light chain variable region set out in SEQ ID NO: 10.

14. The method of any one of the preceding claims, wherein the anti-CD30 antibody of the anti-CD30 antibody drug conjugate is a monoclonal anti-CD30 antibody.

15. The method of any one of the preceding claims, wherein the anti-CD30 antibody of the anti-CD30 antibody drug conjugate is a chimeric AC10 antibody.

16. The method of any one of the preceding claims, wherein the antibody drug conjugate comprises monomethyl auristatin E and a protease-cleavable linker.

17. The method of claim 16, wherein the protease cleavable linker is comprises a thiolreactive spacer and a dipeptide.

18. The method of claim 16 or 17, wherein the protease cleavable linker consists of a thiolreactive maleimidocaproyl spacer, a valine—citrulline dipeptide, and a p-amino-benzyloxycarbonyl spacer.

19. The method of any one of the preceding claims, wherein the anti-CD30 antibody drug conjugate is brentuximab vedotin.

20. The method of any one of the preceding claims, wherein (i) the anti-PD-1 antibody cross-competes with nivolumab or pembrolizumab for binding to human PD-1; (ii) the anti-PD-1 antibody binds to the same epitope as nivolumab or pembrolizumab; (iii) the anti-PD-1 antibody is nivolumab; or (iv) the anti-PD-1 antibody is pembrolizumab.

21. The method of any one of the preceding claims, wherein the anti-PD-1 antibody is nivolumab or pembrolizumab.

22. The method of any one of the preceding claims, wherein the anti-PD-1 antibody is nivolumab.

23. The method of any one of the preceding claims, wherein the hematologic cancer comprises one or more cells that express PD-L1, PD-L2, or both PD-L1 and PD-L2.

24. The method of any one of the preceding claims, wherein the hematologic cancer is a CD30-expressing cancer and the CD30 expression is ≥10%.

25. The method of claim 24, wherein the CD30 expression is measured by a FDA approved test.

26. The method of any one of the preceding claims, wherein the anti-CD30 antibody drug conjugate is brentuximab vedotin and is administered at 1.2 mg/kg, and the anti-PD-1 antibody is nivolumab and is administered at 240 mg/dose.

27. The method of any one of the preceding claims, wherein the anti-CD30 antibody drug conjugate is brentuximab vedotin and is administered at 1.2 mg/kg, and the anti-PD-1 antibody is pembrolizumab and is administered at a dose of 1-2 mg/kg, or 100-300 mg.

28. The method of claim 26 or 27, wherein doxorubicin is administered at dose of 25 mg/m², and dacarbazine is administered at a dose of 375 mg/m².

29. The method of any one of the preceding claims, further comprising administering a granulopoiesis stimulating factor.

30. The method of claim 29, wherein the granulopoiesis stimulating factor is administered prophylactically from 1 day to 7 days after administration of anti-CD30 antibody drug conjugate.

31. The method of any one of claim 29 or 30, wherein the granulopoiesis stimulating factor is administered from 2 days to 5 days after the administration of anti-CD30 antibody drug conjugate.

32. The method of any one of claims 29 to 31, wherein the granulopoiesis stimulating factor is administered about 24 hours to about 36 hours after administration of anti-CD30 antibody drug conjugate.

33. The method of any one of claims 29 to 32, wherein the granulopoiesis stimulating factor is a granulocyte-colony stimulating factor (GCSF).

34. The method of claim 33, wherein the GCSF is a long-acting GCSF or a non long-acting GCSF.

35. The method of claim 33 or 34, wherein the GCSF is long-acting GCSF, and is administered 1 day or 2 days after the administration of anti-CD30 antibody drug conjugate.

36. The method of claim 35, wherein the G-CSF is administered about 24 hours to about 36 hours after administration of anti-CD30 antibody drug conjugate.

37. The method of claim 33 or 34, wherein the GCSF is not long acting, and is administered 1, 2, 3, 4, 5, 6 or 7 days after the administration of anti-CD30 antibody drug conjugate.

38. The method of any one of claims 29 to 37, wherein the granulopoiesis stimulating factor is administered in a dose range from 5 to 10 mcg/kg/day, or 300 to 600 mcg/day, or 6 mg/dose.

39. The method of any one of claims 29 to 38, wherein the granulopoiesis stimulating factor is administered to a subject that has not received anti-CD30 antibody drug conjugate therapy previously.

40. The method of any one of claims 29 to 39, wherein the subject has not experienced treatment-emergent grade 3-4 neutropenia after anti-CD30 antibody drug conjugate administration.

41. The method of any one of claims 29 to 40, wherein the granulopoiesis stimulating factor is given intravenously or subcutaneously.

42. The method of any one of claims 29 to 41, wherein the granulopoiesis stimulating factor is given in a single dose or multiple doses.

43. The method of any one of the preceding claims, wherein if the subject exhibits Grade 3 or Grade 4 neuropathy, the administration of anti-CD30 antibody drug conjugate therapy is withheld until peripheral neuropathy decreases to Grade 2 or less and then 0.9 mg/kg anti-CD30 antibody drug conjugate therapy is administered.

44. The method of claim 43, wherein the neuropathy is motor neuropathy or sensory neuropathy.

45. The method of any one of the preceding claims, wherein the dose of anti-CD30 antibody drug conjugate is delayed by one week if peripheral neuropathy appears, and therapy is continued when the neuropathy is resolved or determined to be Grade 1 or less.

46. The method of any one of the preceding claims, wherein the hematologic cancer is selected from the group consisting of classical Hodgkin Lymphoma, non-Hodgkin Lymphoma, cutaneous T-cell lymphoma (CTCL), and anaplastic large cell lymphoma (ALCL).

47. The method of claim 46, wherein the hematologic cancer is classical Hodgkin Lymphoma.

48. The method of claim 47, wherein the hematologic cancer is a stage IIA with bulky disease, stage IIB, stage III or stage IV classical Hodgkin Lymphoma.

49. The method of claim 46, wherein the anaplastic large cell lymphoma (ALCL) is a systemic anaplastic large cell lymphoma (sALCL).

50. The method of claim 46, wherein the cutaneous T-cell lymphoma (CTCL) is a mycosis fungoides (MF).

51. The method of claim 50, wherein the mycosis fungoides (MF) is a CD30-positive mycosis fungoides (MF).

52. The method of claim 46, wherein the cutaneous T-cell lymphoma (CTCL) is a primary cutaneous anaplastic large cell lymphoma (pcALCL).

53. The method of claim 52, wherein the subject has received prior systemic therapy.

54. The method of any one of claims 45 to 53, wherein the hematologic cancer of the subject has not been treated with a checkpoint inhibitor.

55. The method of any one of the preceding claims, wherein the subject is an adult patient.


SEQUENCE LISTING (16 sequences)

SEQ ID NO: 1 - DNA, 351 bp, Mus musculus
  CDS (1)..(351)
cagatccagctgcagcagtctggacctgaggtggtgaagcctggggcttcagtgaagatatcctgcaaggcttctggctacaccttcactgactactatataacctgggtgaagcagaagcctggacagggacttgagtggattggatggatttatcctggaagcggtaatactaagtacaatgagaagttcaagggcaaggccacattgactgtagacacatcctccagcacagccttcatgcagctcagcagcctgacatctgaggacactgctgtctatttctgtgcgaactatggtaactactggtttgcttactggggccaagggactcaggtcactgtctctgca

SEQ ID NO: 2 - protein, 117 aa, Mus musculus
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 3 - DNA, 15 bp, Mus musculus
gactactatataacc

SEQ ID NO: 4 - protein, 5 aa, Mus musculus
DYYIT

SEQ ID NO: 5 - DNA, 51 bp, Mus musculus
tggatttatcctggaagcggtaatactaagtacaatgagaagttcaagggc

SEQ ID NO: 6 - protein, 17 aa, Mus musculus
WIYPGSGNTKYNEKFKG

SEQ ID NO: 7 - DNA, 24 bp, Mus musculus
tatggtaactactggtttgcttac

SEQ ID NO: 8 - protein, 8 aa, Mus musculus
YGNYWFAY

SEQ ID NO: 9 - DNA, 333 bp, Mus musculus
  CDS (1)..(333)
gacattgtgctgacccaatctccagcttctttggctgtgtctctagggcagagggccaccatctcctgcaaggccagccaaagtgttgattttgatggtgatagttatatgaactggtaccaacagaaaccaggacagccacccaaagtcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtggacgttcggtggaggcaccaagctggaaatcaaa

SEQ ID NO: 10 - protein, 111 aa, Mus musculus
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 11 - DNA, 45 bp, Mus musculus
aaggccagccaaagtgttgattttgatggtgatagttatatgaac

SEQ ID NO: 12 - protein, 15 aa, Mus musculus
KASQSVDFDGDSYMN

SEQ ID NO: 13 - DNA, 21 bp, Mus musculus
gctgcatccaatctagaatct

SEQ ID NO: 14 - protein, 7 aa, Mus musculus
AASNLES

SEQ ID NO: 15 - DNA, 27 bp, Mus musculus
cagcaaagtaatgaggatccgtggacg

SEQ ID NO: 16 - protein, 9 aa, Mus musculus
QQSNEDPWT